(19) United States
(12) Patent Application Publication
(10) Pub. No.: US 2018/0078123 A1
(43) Pub. Date: Mar. 22, 2018

(54) IMAGE PROCESSING DEVICE, ENDOSCOPE DEVICE, AND IMAGE PROCESSING METHOD

(71) Applicant: OLYMPUS CORPORATION, Tokyo (JP)

(72) Inventor: Asako SEKI, Tokyo (JP)

(73) Assignee: OLYMPUS CORPORATION, Tokyo (JP)

(21) Appl. No.: 15/827,129

(22) Filed: Nov. 30, 2017

Related U.S. Application Data
(63) Continuation of application No. PCT/JP2015/066071, filed on Jun. 3, 2015.

(51) Int. Cl. A61B 1/00; G06T 5/50; G06T 7/00

(52) U.S. Cl. CPC A61B 1/00186; G06T 5/50; A61B 5/1032; G06T 7/0014; A61B 1/00009

(57) ABSTRACT

An image processing device includes a processor including hardware, the processor implements an image acquisition process for acquiring a plurality of images including a first image and a second image; a filter process for extracting first to N-th frequency components using first to N-th bandpass filters; correlation calculation for obtaining first to N-th correlation calculation results at a target pixel by performing correlation calculation with an i-th frequency component in the first image and the i-th frequency component in the second image; a reliability calculation process for obtaining reliability of each of the correlation calculation results obtained; a weight setting process for setting a weight of each of the correlation calculation results using the reliability; and amount of disparity calculation for obtaining an amount of disparity using the weight and the first to the N-th correlation calculation results.

### CROSS REFERENCE TO RELATED APPLICATION

This application is a continuation of International Patent Application No. PCT/JP2015/066071, having an international filing date of Jun. 3, 2015, which designated the United States, the entirety of which is incorporated herein by reference.

### BACKGROUND

Stereo matching is a widely known method for obtaining depth (depth information, distance information) based on an image. As disclosed in C. Rhemann, A. Hosni, M. Bleyer, C. Rother, M. Gelautz: “Fast Cost-Volume Filtering for Visual Correspondence and Beyond”; Talk: IEEE Conference on Computer Vision and Pattern Recognition (CVPR) 2011, Colorado Springs; Jun. 21, 2011-Jun. 23, 2011; in: “IEEE”, a known method uses information acquired through some processing on an original image in stereo matching to perform correlation calculation.

C. Rhemann et al. discloses a method for calculating single cost by combining a result of correlation calculation using a pixel value (luminance signal) of an original image and a result of correlation calculation using a gradient signal of the original image.

JP-A-2010-16580 and JP-A-2003-269917 disclose a method for calculating reliability of a cost function through correlation calculation.

### SUMMARY

According to one aspect of the invention, there is provided an image processing device comprising a processor comprising hardware,

the processor being configured to implement:

an image acquisition process for acquiring a plurality of images at least including a first image and a second image;

a filter process for extracting first to N-th (N being an integer equal to or larger than 2) frequency components from each of the first image and the second image, using first to N-th bandpass filters which pass first to N-th frequency bandwidths respectively;

correlation calculation for obtaining first to N-th correlation calculation results by performing correlation calculation with an i-th (i being an integer satisfying 1≦i≦N) frequency component in the first image and the i-th frequency component in the second image to obtain an i-th correlation calculation result at a target pixel;

a reliability calculation process for obtaining reliability of each of the first to the N-th correlation calculation results obtained;

a weight setting process for setting a weight of each of the first to the N-th correlation calculation results using the reliability; and

amount of disparity calculation for obtaining an amount of disparity between the first image and the second image at the target pixel using the set weight and the first to the N-th correlation calculation results.

According to another aspect of the invention, there is provided an endoscope device comprising the above image processing.

According to another aspect of the invention, there is provided an image processing method comprising:

performing a process for acquiring a plurality of images at least including a first image and a second image;

extracting first to N-th (N being an integer equal to or larger than 2) frequency components from each of the first image and the second image, using first to N-th bandpass filters which pass first to N-th frequency bandwidths respectively;

obtaining first to N-th correlation calculation results by performing correlation calculation with an i-th (i being an integer satisfying 1≦i≦N) frequency component in the first image and the i-th frequency component in the second image to obtain an i-th correlation calculation result at a target pixel;

obtaining reliability of each of the first to the N-th correlation calculation results obtained;

setting a weight of each of the first to the N-th correlation calculation results using the reliability; and

obtaining an amount of disparity between the first image and the second image at the target pixel using the set weight and the first to the N-th correlation calculation results.

### BRIEF DESCRIPTION OF THE DRAWINGS

FIG. 1 illustrates an example of a configuration of an image processing device according to an embodiment.

FIG. 2 is a schematic view illustrating a process according to the present embodiment.

FIG. 3 illustrates an example of a configuration of the image processing device according to a first embodiment.

FIG. 4 is a diagram illustrating image shift processing.

FIG. 5 is a diagram illustrating a process for obtaining a cost function.

FIG. 6A to FIG. 6E are charts illustrating relations between the shape of the cost function and reliability.

FIG. 7A and FIG. 7B are charts illustrating relations between the shape of the cost function and reliability.

FIG. 8 is a diagram illustrating a method for obtaining reliability from a plurality of indices.

FIG. 9 illustrates an example of a process for setting weight from reliability.

FIG. 10 is a diagram illustrating a process for obtaining an amount of disparity from the cost function.

FIG. 11 is a schematic view illustrating a process according to the first embodiment.

FIG. 12 is a flowchart illustrating a process according to the first embodiment.

FIG. 13 illustrates an example of a relation between passbands of a plurality of bandpass filters.

FIG. 14 illustrates an example of a configuration of an image processing device according to a second embodiment.

FIG. 15 is a schematic view illustrating a process according to the second embodiment.

FIG. 16 is a flowchart illustrating the process according to the second embodiment.

### DESCRIPTION OF EXEMPLARY EMBODIMENTS

According to one embodiment of the invention, there is provided an image processing device comprising a processor comprising hardware,

the processor being configured to implement:

an image acquisition process for acquiring a plurality of images at least including a first image and a second image;

a filter process for extracting first to N-th (N being an integer equal to or larger than 2) frequency components from each of the first image and the second image, using first to N-th bandpass filters which pass first to N-th frequency bandwidths respectively;

correlation calculation for obtaining first to N-th correlation calculation results by performing correlation calculation with an i-th (i being an integer satisfying 1≦i≦N) frequency component in the first image and the i-th frequency component in the second image to obtain an i-th correlation calculation result at a target pixel;

a reliability calculation process for obtaining reliability of each of the first to the N-th correlation calculation results obtained;

a weight setting process for setting a weight of each of the first to the N-th correlation calculation results using the reliability; and

amount of disparity calculation for obtaining an amount of disparity between the first image and the second image at the target pixel using the set weight and the first to the N-th correlation calculation results.

In the image processing device,

the processor may obtain a corresponding pixel on the second image, the corresponding pixel being a pixel shifted from the target pixel on the first image by a set shift amount, and

the processor may obtain the i-th correlation calculation result at the target pixel based on information corresponding to the target pixel in the i-th frequency component in the first image and based on information corresponding to the corresponding pixel in the i-th frequency component in the second image.

In the image processing device,

the first to the N-th correlation calculation results may be first to N-th cost functions, and

each of the first to the N-th cost functions may be information in which a cost value, calculated by the correlation calculation, and the shift amount are associated with each other.

In the image processing device,

the processor may obtain a combined cost function by performing a weighted sum process using the weight, set by the weight setting process, on the first to the N-th cost functions, and may obtain the among of disparity based on the combined cost function.

In the image processing device,

the first to the N-th correlation calculation results may be first to N-th amounts of disparity, and each of the first to the N-th amounts of disparity may be an amount of disparity obtained for a corresponding one of the frequency components based on a cost function obtained by associating a cost value, calculated by the correlation calculation, with the shift amount.

In the image processing device,

the processor may obtain the amount of disparity by performing a weighted sum process, using the weight set by the weight setting process, on the first to the N-th amounts of disparity.

In the image processing device,

the processor may obtain the reliability based on information on a difference or a ratio between a first local minimum value and a second local minimum value, the first local minimum value and the second local minimum value respectively being a smallest one and a second smallest one of local minimum values of the cost value, or based on information on a difference or a ratio between a first local maximum value and a second local maximum value, the first local maximum value and the second local maximum value respectively being a largest one and a second largest one of local maximum values of the cost value.

In the image processing device,

the processor may obtain the reliability based on a steepness of a change in the cost value relative to a change in the shift amount, within a given shift amount range including a local maximum value or a local minimum value of the cost value.

In the image processing device,

the first to the N-th bandpass filters may have resonance frequencies f₁ to f_{N} satisfying fₖ<fₖ₊₁ (k being an integer satisfying 1≦k≦N−1), and

fHₖ≧fLₖ₊₁ may be satisfied where fHₖ represents an upper cutoff frequency of a k-th bandpass filter in the first to the N-th bandpass filters and fLₖ₊₁ represents a lower cutoff frequency of a k+1th band pass filter.

In the image processing device, the processor may obtain, when the reliability of each of the first to the N-th correlation calculation results is smaller than a given threshold value, the amount of disparity at the target pixel is obtained based on the amount of disparity of a pixel other than the target pixel.

In the image processing, the processor may set the weight to be 0 for a correlation calculation result, the reliability of which is smaller than a given threshold value, among the first to the N-th correlation calculation results.

According to another embodiment of the invention, there is provided an endoscope device comprising the above image processing device.

In the endoscope device, the first image and the second image each may be an in vivo image.

According to another embodiment of the invention, there is provided an image processing method comprising:

performing a process for acquiring a plurality of images at least including a first image and a second image;

extracting first to N-th (N being an integer equal to or larger than 2) frequency components from each of the first image and the second image, using first to N-th bandpass filters which pass first to N-th frequency bandwidths respectively;

obtaining first to N-th correlation calculation results by performing correlation calculation with an i-th (i being an integer satisfying 1≦i≦N) frequency component in the first image and the i-th frequency component in the second image to obtain an i-th correlation calculation result at a target pixel;

obtaining reliability of each of the first to the N-th correlation calculation results obtained;

setting a weight of each of the first to the N-th correlation calculation results based on the reliability; and

obtaining an amount of disparity between the first image and the second image at the target pixel using the set weight and the first to the N-th correlation calculation results.

The present embodiment will be described below. The present embodiment described below is not intended to unduly limit the scope of the present invention described in the appended claims. Not all the components described in the present embodiment are essential for the present invention.

### 1. Method According to the Present Embodiment

First of all, a method according to the present embodiment is described. As described above, in C. Rhemann et al., correlation calculation using a luminance signal based on an original image (input image, captured image) and correlation calculation using a gradient signal based on the original image are performed. Then, an amount of disparity is obtained by using results of these two calculations. The gradient signal is obtained by extracting components corresponding to relatively high spatial frequencies from an original image. Thus, an amount of disparity is expected to be more accurately obtained than in a process using only the original image (only the luminance signal), when the subject has a feature well represented by a high frequency band (high frequency). In the description below in this specification and the like, the term “frequency” represents spatial frequency unless stated otherwise.

For example, an input image with high frequency components emphasized involves enhanced noise. This enhanced noise has a negative impact on a result of the correlation calculation using the luminance signal and a result of the correlation calculation using the gradient signal, resulting in a matching accuracy extremely compromised. Different subjects have different features, and thus the gradient signal is not always necessarily appropriate for obtaining an amount of disparity.

More specifically, a signal (information and a spatial frequency band) used for calculating an amount of disparity preferably well represents the feature of the subject or is preferably less affected by noise. In this context, effectiveness of the gradient signal used in C. Rhemann et al. is not guaranteed. Specifically, this signal can achieve a suitable process (accurate stereo matching) in the case where the feature of the subject appears in a high frequency band, but is difficult to achieve the suitable process in the case where the feature of the subject appears in a relatively low spatial frequency band (low frequency band or a middle frequency band), or in a case involving a large amount of noise in a high frequency band.

Setting a signal with components in a wide frequency band set as a process target would not be an effective solution to achieve higher accuracy. Logically, the process target as a signal including components in a wide frequency band is likely to include frequency components well representing the feature of the subject, but is also likely to include frequency components not representing the feature of the subject. Thus, the feature of the subject becomes difficult to discern. In an extreme case, the correlation calculation may be performed by using a signal obtained by extracting components over the entire frequency band (with no frequency band excluded) from an input image; however, it can be readily understood that this is nothing different from the correlation calculation directly using the input image (luminance signal), and thus does not facilitate an attempt to improve the accuracy of the calculation of an amount of disparity.

All things considered, an amount of disparity should be accurately obtained through correlation calculation on components in an appropriate frequency band extracted from an input image. Unfortunately, the “appropriate frequency band” depends on the feature of a subject. Specifically, the frequency band of the components to be extracted varies when a subject as an image capturing target changes, and may also vary for the same object when a status of a light source changes or an optical condition involving a lens system, an image sensor, and the like changes.

Thus, when an amount of disparity is to be obtained from a predetermined input image, the frequency band suitable for the input image is difficult to be set in advance. The frequency band of components to be extracted from the input image may be fixed to a given band. As in the case of C. Rhemann et al., such a configuration might result in an accurate amount of disparity obtained or might be difficult to calculate an amount of disparity accurately, and thus lacks versatility.

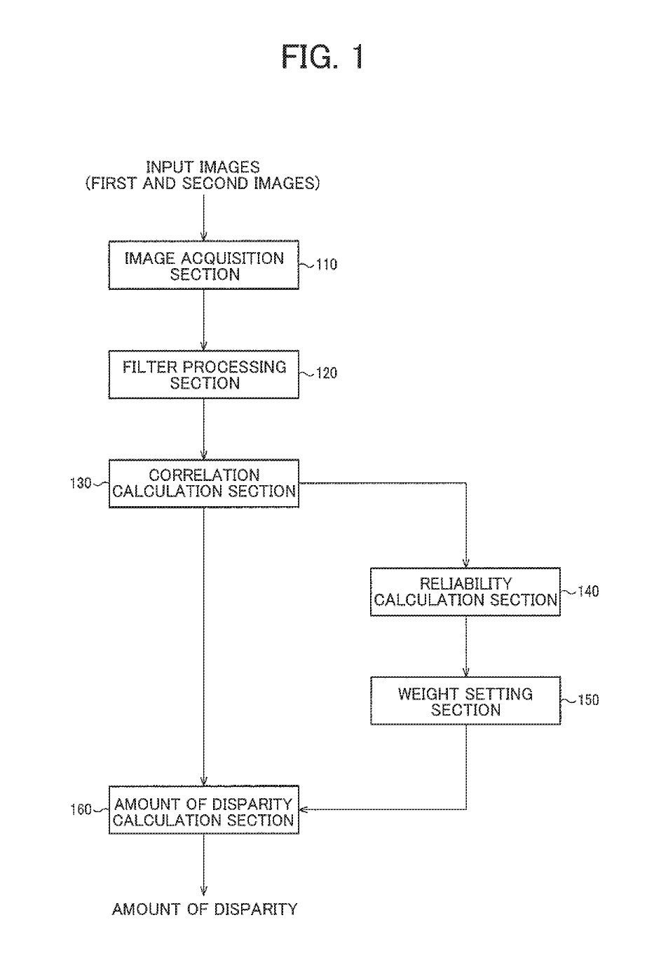

In view of the above, the present applicant proposes a method of adaptively changing a frequency band used for calculating an amount of disparity (more specifically, a frequency with a larger weight in the calculation) depending on the situation. Specifically, as illustrated in FIG. 1, an image processing device according to the present embodiment includes: an image acquisition section 110 acquiring a plurality of images at least including a first image and a second image; a filter processing section 120 extracting first to N-th (N being an integer equal to or larger than 2) frequency components from each of the first image and the second image, using first to N-th bandpass filters BPF1 to BPFN which pass first to N-th frequency bandwidths respectively; a correlation calculation section 130 obtaining first to N-th correlation calculation results by performing correlation calculation with an i-th (i being an integer satisfying 1≦i≦N) frequency component in the first image and the i-th frequency component in the second image to obtain an i-th correlation calculation result at a target pixel; a reliability calculation section 140 obtaining reliability of each of the first to the N-th correlation calculation results obtained; a weight setting section 150 setting a weight of each of the first to the N-th correlation calculation results using the reliability; and an amount of disparity calculation section 160 obtaining an amount of disparity between the first image and the second image at the target pixel using the set weight and the first to the N-th correlation calculation results.

The plurality of images acquired by the image acquisition section 110 are disparity images having disparity therebetween. More specifically, the first image may be a reference image serving as a reference for calculating the amount of disparity, and the second image may be a search image for detecting a shifted pixel amount from the reference image.

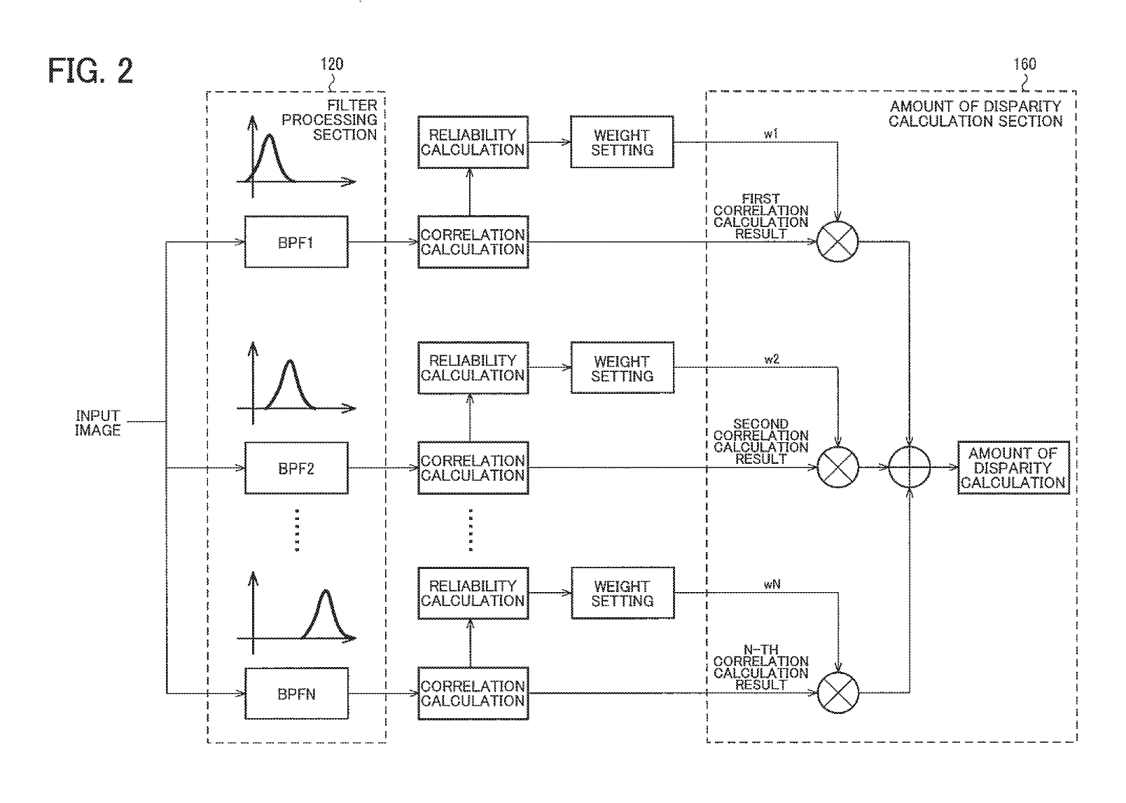

FIG. 2 is a schematic view illustrating a method according to the present embodiment. As illustrated in FIG. 2, in the present embodiment, N bandpass filters BPF1 to BPFN are applied to first and second images that are input images to extract N frequency components from each of the images. Then, correlation calculation is performed on each of the components to obtain N correlation calculation results. The N correlation calculation results are appropriately weighted and then combined, and thus an amount of disparity is obtained.

In this process, a frequency band with a higher reliability may be provided with a larger weight so that the combining can be performed with correlation calculation results obtained with frequency bands representing the feature having larger weights (higher contributions). Thus, even when the feature of the subject is unknown and thus appropriate frequency bands are unknown at the point where the process starts, appropriate components can be selected (more specifically, provided with a larger weight) by actually obtaining the correlation calculation results for the N frequency components. Thus, a versatile and accurate process can be achieved. In other words, stereo matching robust against the change in the feature of the subject can be achieved.

In first and second embodiments described below, an image processing device is described. However, the method according to the embodiments is not limited to this, and may be applied to an endoscope device including the image processing device. In this case, the plurality of images, including the first and the second images, may be in vivo images obtained by image capturing in a living body.

The endoscope has an image capturing section (insert section) inserted into an image capturing target, and thus is difficult to use sunlight, room light, and the like as a light source. Thus, the image is captured by using light emitted from a light source section provided to an end or the like of the image capturing section. Thus, an endoscope image is likely to be affected by a state of the light source (for example, relative positions and orientations of the light source and the subject). Thus, a frequency band representing the feature of the subject is likely to vary.

The frequency band representing the feature of the subject is particularly likely to vary when the endoscope device is a medical endoscope device and the first and the second images area in vivo images. Specifically, in many cases, the in vivo images include various subjects such as blood vessels, a wall of an internal organ, a lesion area, bubbles, and residues. The frequency band representing the feature varies among the subjects, and thus the frequency band representing the feature of the subject is less likely to be uniform.

Furthermore, the feature of the subject changes when the subject surface is wet with liquid, and also when pigments are sprayed to facilitate observation by a user (physician). In Narrow Band Imaging (NBI) using light in a wavelength band narrower than RGB wavelength bands of general white light, the color tone of the subject changes from that in an RGB image, and thus the frequency band representing the feature changes. As described above, the feature in the in the vivo image is artificially changed to achieve higher visibility in many cases, and thus the frequency band suitable for the stereo matching largely varies for the same image capturing subject.

Thus, the appropriate frequency band is extremely difficult to set in advance for the in vivo image captured with the endoscope device, and thus a highly versatile (robust) method according to the present invention should be highly effective for such a situation.

The method according to the present embodiment may be applied to an image processing method (a method for operating and controlling an image processing device). More specifically, the method according to the present embodiment may be applied to an image processing method (a method for operating and controlling an image processing device) including: by the image acquisition section 110, performing a process for acquiring a plurality of images at least including a first image and a second image; by the filter processing section 120, extracting first to N-th (N being an integer equal to or larger than 2) frequency components from each of the first image and the second image, based on first to N-th bandpass filters which pass first to N-th frequency bandwidths respectively; by the correlation calculation section 130, obtaining first to N-th correlation calculation results by performing correlation calculation with an i-th (i being an integer satisfying 1≦i≦N) frequency component in the first image and the i-th frequency component in the second image to obtain an i-th correlation calculation result at a target pixel; by the reliability calculation section 140, obtaining reliability of each of the first to the N-th correlation calculation results obtained; by the weight setting section 150, setting a weight of each of the first to the N-th correlation calculation results using the reliability; and by the amount of disparity calculation section 160, obtaining an amount of disparity between the first image and the second image at the target pixel using the set weight and the first to the N-th correlation calculation results.

The first and the second embodiment are described in detail below. The first embodiment and the second embodiment are different from each other in the correlation calculation result obtained with each frequency components. As will be described in detail below, a method according to the first embodiment obtains a cost function as the correlation calculation result, and a method according to the second embodiment obtains an amount of disparity (not an amount of disparity finally obtained, but an amount of disparity obtained with each frequency component) as the correlation calculation result.

### 2. First Embodiment

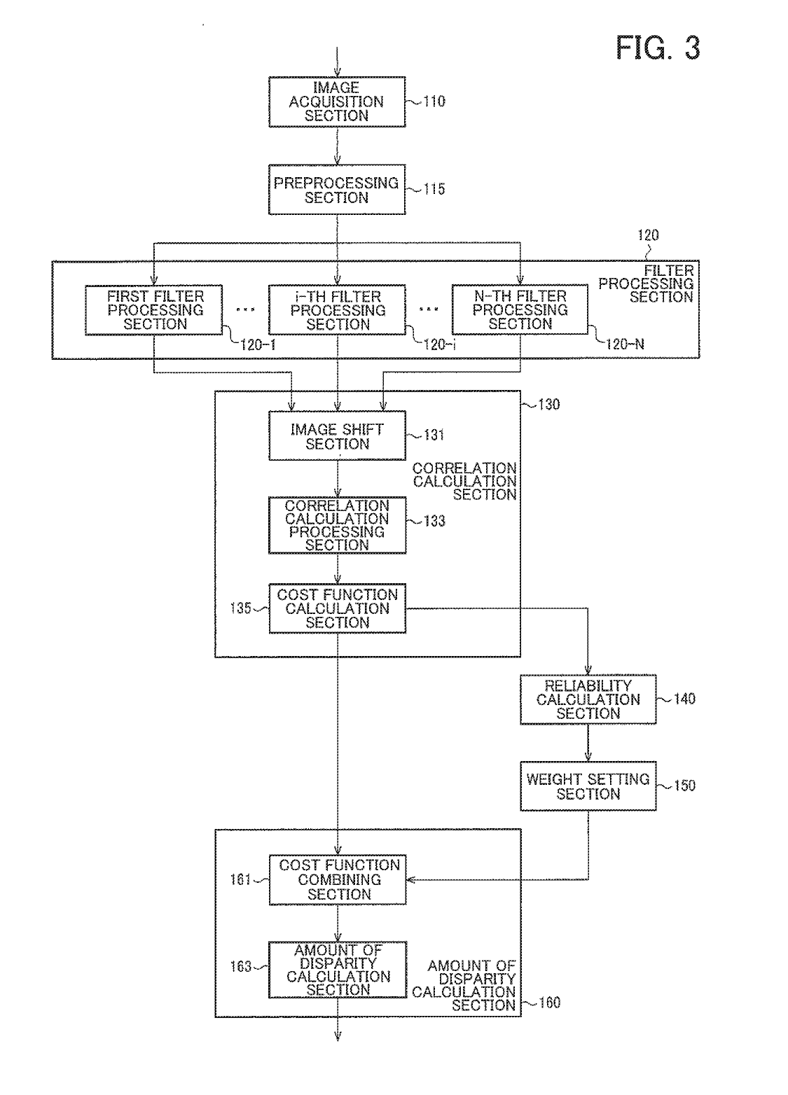

FIG. 3 illustrates an example of a configuration of an image processing device according to the first embodiment. This image processing device includes the image acquisition section 110, a preprocessing section 115, the filter processing section 120, the correlation calculation section 130, the reliability calculation section 140, the weight setting section 150, and the amount of disparity calculation section 160.

The filter processing section 120 includes first to N-th filter processing sections 120-1 to 120-N. The correlation calculation section 130 includes an image shift section 131, a correlation calculation processing section 133, and a cost function calculation section 135. The amount of disparity calculation section 160 includes a cost function combining section 161 and an amount of disparity calculation section 163. The image processing device is not limited to the configuration illustrated in FIG. 3, and various modifications may be made with the components in the figure partially omitted or unillustrated components additionally provided.

A flow of a stereo matching process (a process for obtaining an amount of disparity) according to the present embodiment is described with each section described in detail. The image acquisition section 110 acquires two or more input images (disparity images) having disparity. In the description below, a method of obtaining an amount of disparity between the two images are described. However, this is merely for the sake of description, and three or more images may be processed. When three or more input images are processed, one of the input images may be determined as the reference image. The correlation calculation may be performed with the remaining plurality of images and the reference image, and the results of the calculation may be combined to obtain an amount of disparity.

The preprocessing section 115 performs preprocessing on the input images. For example, when the input images include a large amount of noise, a noise reduction process may be performed as the preprocessing. When the stereo matching is performed, epipolar lines may be matched between the images in advance through camera calibration or the like. Thus, a search range described later can be limited to a single direction (horizontal direction), whereby a calculation amount can be reduced. The preprocessing section 115 may perform correction processing and the like using a parameter acquired by camera calibration as appropriate.

The filter processing section 120 executes filter processing using bandpass filters on input images (input image subjected to the preprocessing by the preprocessing section 115 as appropriate) input to the image acquisition section 110. The filter processing section 120 includes at least two filters (bandpass filters) with different passbands. In this example, the filter processing section 120 includes the first to the N-th bandpass filters BPF1 to BPFN with different passbands, and an i-th (i being an integer satisfying 1≦i≦N) filter processing section 120-i applies an i-th bandpass filter to the input images.

The passbands of the plurality of bandpass filters may overlap. The bandpass filters may not have a uniform bandwidth. The filter is not limited to the bandpass filter, and may be a band emphasis filter. Specifically, a gain in a passband may not be 1 and may be a value larger than 1 (or may be a value smaller than 1). The configuration of each bandpass filter may be modified in various ways.

With the first to the N-th bandpass filters applied to the first image and the second image, first to N-th frequency components are extracted from the first image and first to N-th frequency components are also extracted from the second image. Thus, the number of signals output from the filter processing section 120 is (the number of input images)×(the number of bandpass filters).

A process described below includes: a process for a set of a first frequency component in the first image and a first frequency component in the second image; a process for a set of a second frequency component in the first image and a second frequency component in the second image; . . . and a process for a set of an N-th first frequency component in the first image and an N-th frequency component in the second image. Specifically, the correlation calculation section 130, the reliability calculation section 140, and the weight setting section 150 execute processes for each frequency component. Thus, the same process is repeated for the number of times corresponding to the number of frequency components (the number of bandpass filters).

The description is given below based on a single given frequency component, to simplify the description.

The correlation calculation section 130 performs correlation calculation to calculate correlation between the two images (the i-th frequency component in the first image and the i-th frequency component in the second image). Processes executed by the image shift section 131, the correlation calculation processing section 133, and the cost function calculation section 135, in the correlation calculation section 130, are described in detail below.

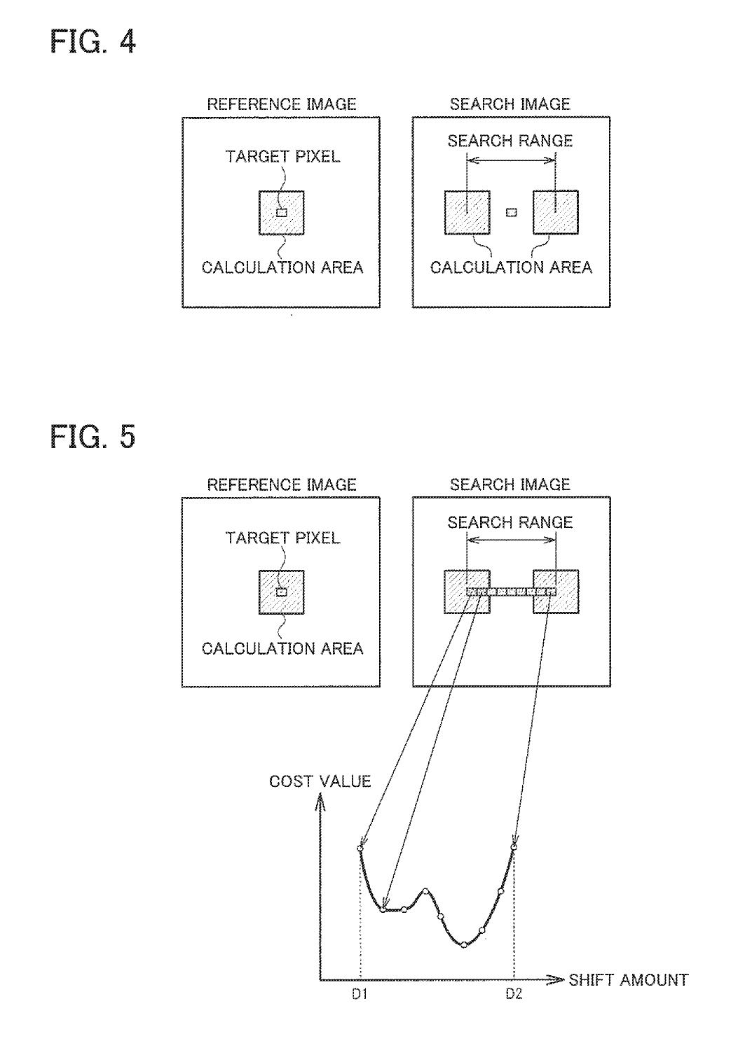

The correlation calculation section 130 uses a target pixel in one of the images (first image) as a reference and performs the correlation calculation within a range of a search range D1 to D2 in the other one of the images (the second image). The image shift section 131 shifts the second image by a set shift amount k. In the present embodiment, the image shift section 131 shifts the second image, serving as the search image, at a given interval (for example, at an interval of one pixel) within the range D1 to D2. The specific method for the shifting is not limited to a single method. For example, as illustrated in FIG. 4, a calculation area (an area that is a target of the correlation calculation as described later) may be shifted by a pixel-by-pixel basis, or the entire image may be shifted. The search range set to extend in both a + direction and a − direction relative to a disparity direction (D1<0<D2) in FIG. 4 may be limited to one of the directions.

The correlation calculation processing section 133 performs correlation calculation with a calculation area, on the first image, with the target pixel i at the center, and with a calculation area, on the second image, with a pixel i+k (k representing the shift amount as described above) at the center. The pixel i+k is a pixel shifted from the target pixel within the search range. The correlation calculation performed by the correlation calculation processing section 133 may employ various methods. For example, sum of absolute difference (SAD), Zero-mean Normalized Cross Correlation (ZNCC), or a hamming distance after census transform may be obtained. Through the calculation process performed by the correlation calculation processing section 133, one calculation value is obtained per shift amount. In the present embodiment, one calculation value obtained by the correlation calculation processing section 133 is referred to as a cost value. As used herein, cost (cost value) is an index indicating correlation between two areas to be compared with each other. The description is given below under an assumption that a smaller cost value represents a higher correlation between two areas. However, depending on a matching method employed (for example ZNCC), a larger cost value may represent a higher correlation.

As illustrated in FIG. 5, the cost function calculation section 135 acquires the cost function for each target pixel by using the calculation result obtained by the correlation calculation processing section 133. Specifically, the cost function of the target pixel i is a data sequence representing the correlation calculation results calculated for the target pixel i within the search range. The process may be regarded as a process for associating a cost value with the shift amount k. The cost function calculation section 135 may perform smoothing processing on the cost functions calculated. In such a configuration, the Guided Filter described in C. Rhemann et al. or the like may be used for example.

In the present embodiment, the cost function obtained by the cost function calculation section 135 is output as the correlation calculation result. As described above, the correlation calculation section 130 performs the process on each frequency component. Thus, first to N-th cost functions, as a result of processing the first to the N-th frequency components, are output as the correlation calculation results.

The reliability calculation section 140 calculates the reliability of each correlation calculation result. In the present embodiment, the reliability of each of the first to the N-th cost functions is obtained. In other words, the number of filters (at least two) of the filter processing section 120 corresponds to the number of reliabilities calculated. In the present embodiment, the reliability is calculated based on the cost function (from its shape in a narrow sense). For example, the reliability may be the difference between the local minimum value and the second local minimum value of the cost function (SAD) as in JP-A-2010-16580. Alternatively, the reliability may be a width of a portion around the local minimum value of the cost function in the horizontal axis direction or may be the steepness of the cost function as in JP-A-2003-269917. The reliability calculation method performed by the reliability calculation section 140 is not limited to this. The reliability may also be an edge intensity or the like.

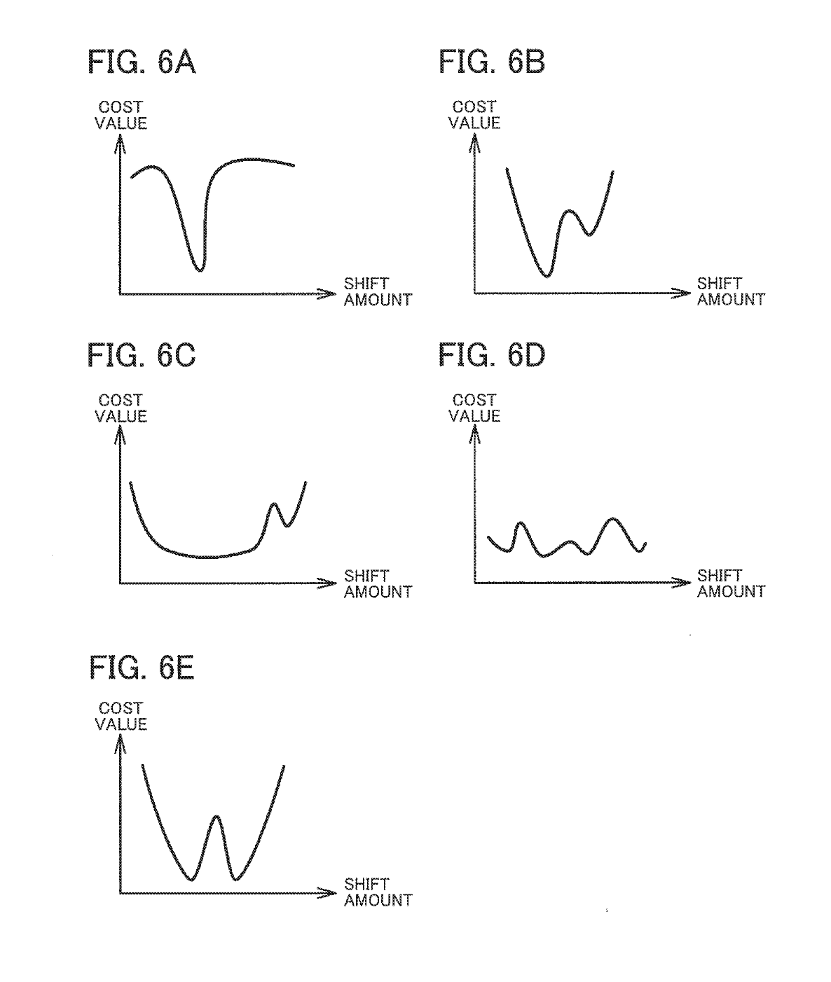

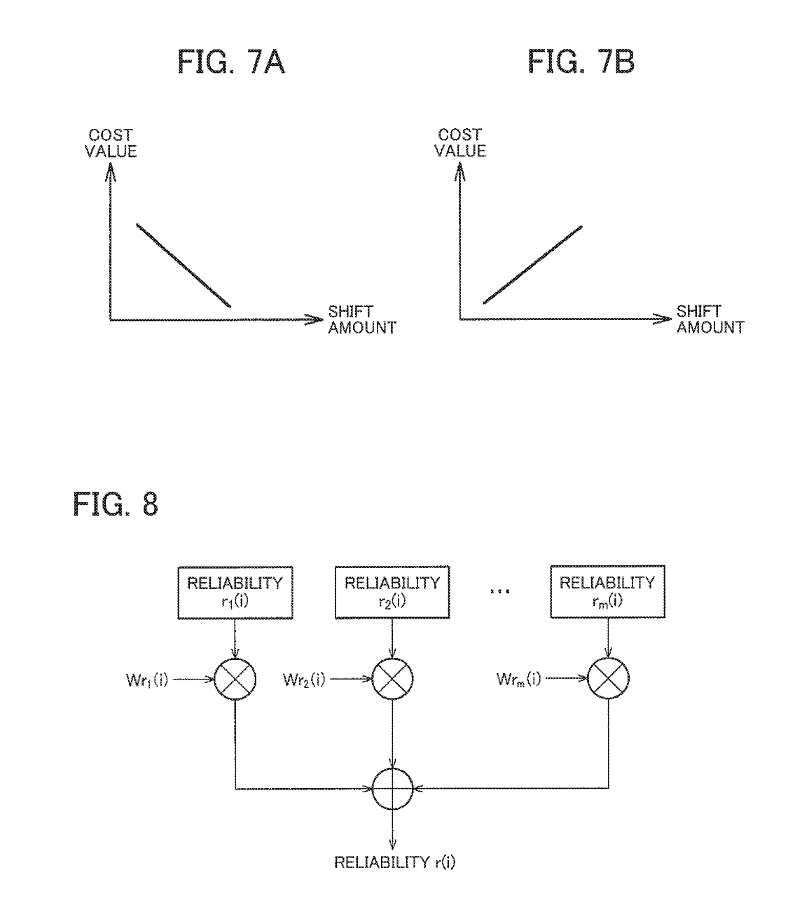

A reliability is calculated to be high for a frequency component with a cost function with the local minimum value clearly determined as illustrated in FIG. 6A and FIG. 6B. On the other hand, a reliability is set to be low for a frequency component with a cost function with no obvious local minimum value, as illustrated in FIG. 6C to FIG. 6E, such as that obtained in a flat area. When the cost value linearly increases or decreases as illustrated in FIG. 7A and FIG. 7B, the amount of disparity at the target pixel is expected to be outside the search range. For example, the example illustrated in FIG. 7A indicates that the amount of disparity to be obtained is more on the + direction side than the end (D2) of the search range on the + direction side. On the other hand, the example illustrated in FIG. 7B indicates that the amount of disparity to be obtained is more on the − direction side than the end (D1) of the search range on the − direction side. Thus, the appropriate amount of disparity cannot be obtained with the cost functions as illustrated in FIG. 7A and FIG. 7B, and thus the reliability is set to be low for such cost functions.

The reliability may be calculated with a single one of the indices described above, and may be calculated with a combination of a plurality of indices. For example, a reliability r of a target cost function may be obtained based on a first reliability r₁ representing a difference between the local minimum value (first local minimum value) and the second local minimum value of the cost function, a second reliability r₂ representing the steepness of the cost function, and a third reliability r₃ representing the edge intensity.

Thus, the reliability calculation section 140 may calculate a reliability r(i) for the i-th frequency component through weighted sum as illustrated in the following Formula (1):

$$\begin{matrix}[ Formula 1 ] & \\ r ( i ) = {\sum }_{j} {wr}_{j} ( i ) \times {r}_{j} ( i ) , & ( 1 )\end{matrix}$$

where i represents the frequency component, j represents each reliability calculation index, and wr represents the weight.

FIG. 8 is a diagram illustrating a process represented by Formula (1) described above. As described above, the reliability may be obtained by using a plurality of indices.

The weight setting section 150 sets (calculates) the weight of each frequency component, based on the reliability calculated by the reliability calculation section 140. The number of weights to be calculated corresponds to the number of frequency components, that is, the number of filters of the filter processing section 120.

Specifically, a larger weight is set for a higher reliability calculated, and a smaller weight is set for a lower reliability calculated. For example, a value as a result of constant multiplication of the reliability using a given coefficient may be set as the weight. Alternatively, normalization may be performed with the sum of (Σr of denominator on the right side) of the reliabilities of all the frequency components as in the Formula (2).

$$\begin{matrix}[ Formula 2 ] & \\ {w}_{i} = α \times \frac{{r}_{i}}{{\sum }_{p} {r}_{p}} & ( 2 )\end{matrix}$$

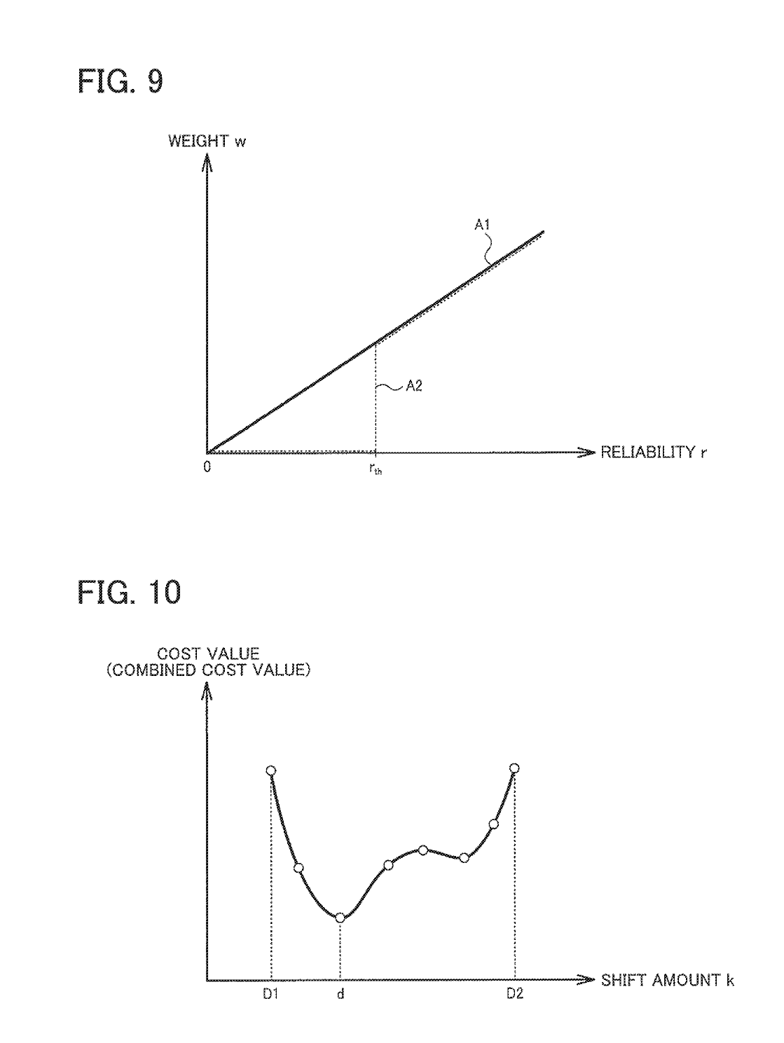

In such a case, the relationship between the reliability and the weight is represented by a straight line denoted with A1 in FIG. 9. In the example represented by Formula (2) described above, the slope of the straight line changes in accordance with the sum of the reliabilities obtained in each frequency band.

The method of obtaining the weight from the reliability is not limited to this. For example, the weight (the weight of the cost function obtained from the frequency component) may be set to be 0 for a frequency component with a reliability lower than a given threshold value. Thus, the process for obtaining an amount of disparity involves no frequency component (cost function) with low reliabilities. The reliability and the weight are in relationship denoted with A2 in FIG. 9, where rₜₕ represents the threshold value of the reliability. Specifically, A2 indicates that the weight is obtained by the constant multiplication of the reliability, equal to or larger than the threshold value. Note that other methods can be used, and only a frequency component with the highest reliability may be used. This process can be regarded as setting weight to be 0 for all the frequency components except for the frequency component with the highest reliability.

The amount of disparity calculation section 160 obtains an amount of disparity at the target pixel using the correlation calculation results (the first to the N-th cost functions in the present embodiment) and the weights.

Specifically, the cost function combining section 161 performs weighted combination for each shift amount, by using first to N-th cost functions C1 to CN calculated by the cost function calculation section 135 and weights w1 to wN of the frequency components calculated by the weight setting section 150. Specifically, a combined cost value Cₜₒₜₐₗ(k) is obtained for the given shift amount k with the following Formula (3),

$$\begin{matrix}[ Formula 3 ] & \\ {C}_{total} ( k ) = a \times {\sum }_{i} {w}_{i} {C}_{i} ( k ) , & ( 3 )\end{matrix}$$

where a represents a given coefficient, k represents the shift amount, i represents each frequency band, Wi represents the weight set for the i-th frequency component, and Ci(k) represents the cost value of the i-th cost function with the shift amount k.

The calculation represented by Formula (3) described above is performed for the search range D1 to D2 while changing the shift amount k whereby a value obtained by combining the cost values in the search range D1 to D2 can be obtained. In other words, the combined cost function Cₜₒₜₐₗ can be obtained by weighted sum of the first to the N-th cost functions with Formula (3) described above. The combined cost function Cₜₒₜₐₗ is information in which the cost value (combined cost value) is associated with each shift amount k, as in the case of each of the first to the N-th cost functions. FIG. 10 illustrates an example of such information.

The amount of disparity calculation section 163 calculates the shift amount k corresponding to one of the cost values (combined cost values), obtained by the cost function combining section 161, with the highest correlation as illustrated in FIG. 10, as the amount of disparity at the target pixel. In the example illustrated in FIG. 10, d represents the amount of disparity to be obtained. Note that the “cost value with a high correlation” changes depending on the type of the correlation calculation. For example, the shift amount with the smallest cost is selected as the amount of disparity, when SAD is employed for the correlation calculation. On the other hand, the shift amount with the largest cost is selected as the amount of disparity, when ZNCC is employed for the correlation calculation.

The amount of disparity may be calculated with subpixel accuracy, by performing parabola fitting or spline interpolation. The first to the N-th cost functions are calculated in a unit of a variation width of the shift amount (for example, a pixel). Thus, the combined cost function is information in which a combined cost value is associated in a pixel-by-pixel basis. Thus, with this information, the amount of disparity is determined and the minimum value is obtained in a pixel order. The actual amount of disparity might be in an order smaller than the pixel order (subpixel order). Thus, the amount of disparity might not be obtained with sufficient accuracy with the pixel order. In view of this, a given interpolation process may be performed so that the amount of disparity can be obtained in a subpixel order. Thus, the amount of disparity can be calculated with higher accuracy.

The processing described above is performed on a single target pixel in the reference image (first image). In the actual case, the process described above may be performed on a plurality of pixels while changing the target pixel. In a narrow sense, with all the pixels on the first image set as the target pixel, the amount of disparity can be obtained for each of the pixel of the first image. In such a case, for example, the image processing device according to the present embodiment outputs information (disparity map) in which an amount of disparity is associated with each pixel on the reference image or information based on the disparity map.

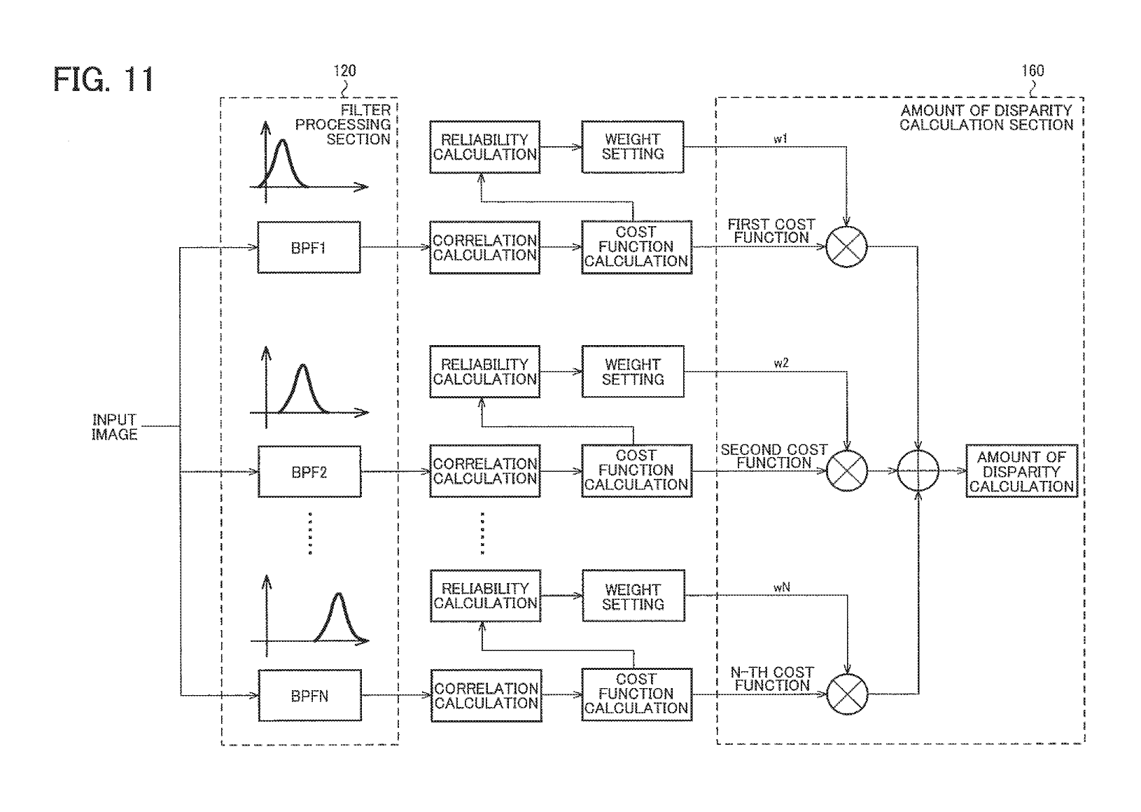

FIG. 11 is a schematic view illustrating the process according to the present embodiment described above. As is clear from the comparison with the schematic view in FIG. 2, in the present embodiment, the cost function is obtained as the correlation calculation result. Thus, the amount of disparity calculation section 160 performs a combination process for combining the cost functions to obtain a combined cost function.

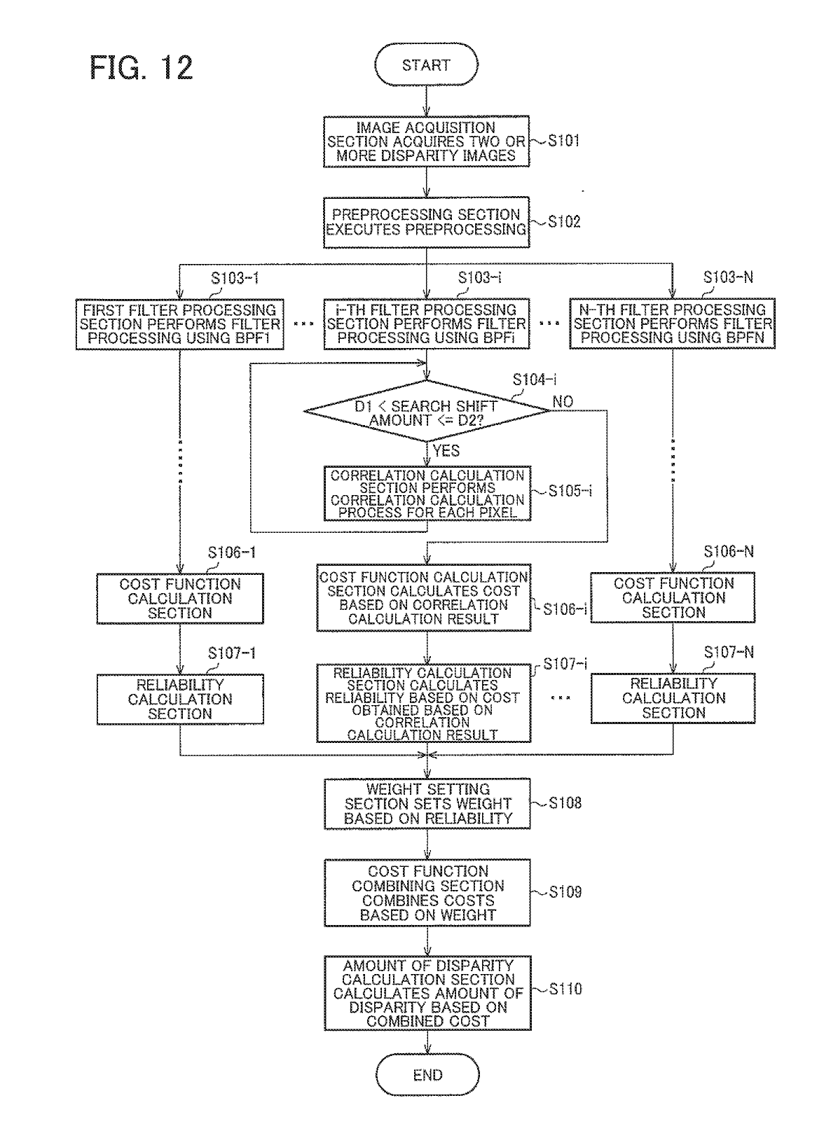

FIG. 12 is a flowchart illustrating the process according to the present embodiment. When the process starts, first of all, the image acquisition section 110 acquires a plurality of input images (S101). Then, the preprocessing such as noise reduction is performed as appropriate on the input images thus acquired (S102).

Next, the filter process using the bandpass filters is performed on each of the plurality of input images (S103). The process in S103 includes a process performed by the first filter processing section 120-1 using the first bandpass filter BPF1 (S103-1), a process performed by the second filter processing section 120-2 using the second bandpass filter BPF2 (S103-2), . . . and a process performed by the N-th filter processing section 120-N using the N-th bandpass filter BPFN (S103-N), as illustrated in FIG. 11 and FIG. 12.

When the i-th frequency component on each input image is obtained in S103-i, the correlation calculation is performed in the search range D1 to D2 while changing the shift amount k (S104-i and S105-i). When the process is completed for the entire search range, No is obtained as a result of the determination in S104-i, and the processing proceeds to S106-i. Specifically, the cost function is obtained based on the cost value obtained with each shift amount k (S106-i) and the reliability of the cost function (frequency component) is calculated from the shape of the cost function thus obtained (S107-i).

The process that is the same as that in S104-i to S107-i is performed on each frequency component. Thus, the first to the N-th cost functions and the reliability of each frequency component are calculated.

After S107-1 to S107-N are completed, the weight is set for each frequency component based on the reliability thus calculated. In this example, the weights are set for the frequency components set by using all the reliabilities as in Formula (2) described above or the like, and thus the process in S108 is performed after S107-1 to S107-N are completed. For example, when the weight of a given frequency component can be set without referring to the reliabilities of the other frequency components, as in the case where the weight is obtained by constant multiplication of the reliability, the process in S108 may be individually performed for each frequency component.

When S108 is completed, the first to the N-th cost functions and the weight corresponding to each of the functions are obtained. Thus, the amount of disparity calculation section 160 uses these values to obtain the combined cost function (S109) and obtains the amount of disparity from the combined cost function (S110). Through the processes described above, the amount of disparity is obtained for a single target pixel. Thus, when there are a plurality of pixels of interest for which the amount of disparity is to be obtained through the processes described above, the processes in FIG. 12 may be repeated for a number of times corresponding to the number of such pixels. For example, the processes in FIG. 12 are repeated for the number of times corresponding to the number of all the pixels on the reference image.

With the cost functions combined with a larger weight provided to a frequency band with a higher reliability as described above, the total cost (combined cost function) is obtained by combining with a cost function, corresponding to a frequency band particularly representing the feature of the subject, provided with a high contribution rate. Thus, the amount of disparity can be more easily determined uniquely from the combined cost function, whereby the amount of disparity can be more accurately calculated.

The method according to the present embodiment is not limited to that described above, and may be modified in various ways.

For example, the filter processing section 120 does not necessarily need to cover the entire band. When the target subject is limited to some extent or when the feature of the target subject is recognized in advance, the band as a target of the filter processing section 120 may be limited. For example, some of the first to the N-th bandpass filters may be prepared but not applied (some of the first to the N-th filter processing sections 120-1 to 120-N may not operate). Thus, a calculation cost can be reduced.

The disparity map obtained by the amount of disparity calculation section 163 may be directly output. However, this should not be construed in a limiting sense, and a certain post processing may be performed on the disparity map obtained. For example, filter processing using a smoothing filter or the like may be executed on the disparity map, and a result of the processing may be output.

When the combined cost function is obtained, information on the combined cost function may be fed back to previous steps. For example, a condition of the process in the previous step may be changed when the combined cost function has a plurality of peaks corresponding to different shift amounts. For example, the feedback may be provided to reduce the size of the calculation area for the correlation calculation.

When none of the reliabilities obtained by the reliability calculation section 140 for the frequency components exceeds the threshold value, the target pixel may be determined as a flat portion, and the amount of disparity at the target pixel may be estimated and interpolated by using the amounts of disparity calculated for other pixels. For example, the amount of disparity at the target pixel may be interpolated based on the amounts of disparity at pixels around the target pixel in the disparity map.

In this interpolation process, not only the disparity map but also information on the input image may be used. When a given area of the input image is estimated to be a captured image of the same area of the same subject, the pixels in the area can be estimated to have the same amount of disparity. Thus, the amount of disparity at the target pixel may be interpolated based on the amounts of disparity at surrounding pixels determined to be in the same area of the same subject as the target pixel, based on color information or the like on the input image.

The threshold value in the process described above may be set in various ways. For example, the threshold value may be calculated and set by using a reliability obtained from an image of a flat object captured in advance. The threshold value thus obtained can be used for determining whether or not the target pixel is in a flat portion, that is, whether the amount of disparity can be accurately obtained with the pixel. Thus, the interpolation process can be executed by using information on other pixels, for the pixel thus determined to be unable to provide an accurate amount of disparity.

In the present embodiment described above, the correlation calculation section 130 obtains a corresponding pixel in the second image that is a pixel as a result of shifting from the target pixel on the first image by a set shift amount. Then, the i-th correlation result at the target pixel is obtained based on information on the target pixel in the i-th frequency component in the first image and based on information corresponding to the corresponding pixel in the i-th frequency component in the second image.

When an area corresponding to a target area in the reference image (first image) is to be detected in the search image (second image), the shift amount serves as a value indicating a shifted amount of the search area from the target area in the horizontal direction in a unit of pixel. Thus, the search area corresponding to the pixel shift amount k from an area with the target pixel (i,j) at the center is an area with (i+k,j) at the center. Thus, the information corresponding to the target area is a calculation area with the target pixel (i,j) at the center, and the information corresponding to the corresponding pixel is a calculation area with the corresponding pixel (i+k,j) at the center.

Thus, when the given shift amount k is set, the correlation calculation can be executed with information based on the first image used for the correlation calculation and information based on the second image appropriately set.

The first to the N-th correlation calculation results are the first to the N-th cost functions, and each of the first to the N-th cost functions is information in which the shift amount k and the cost value calculated by the correlation calculation are associated with each other. The amount of disparity calculation section 160 performs the weighted sum process using the weights set by the weight setting section 150 to the first to the N-th cost functions to obtain the combined cost function Cₜₒₜₐₗ, and obtains the amount of disparity based on this combined cost function.

Thus, the amount of disparity can be obtained by obtaining the cost functions as the correlation calculation result, weighting the cost functions, and then combining the cost functions. The cost function is information obtained based on a predetermined width in the search range D1 to D2 (for example, in order of pixel), and thus the combined cost function is obtained in the same order. Thus, a larger amount of information can be obtained and thus an amount of disparity can be more accurately obtained, compared with a configuration in which amounts of disparity corresponding to frequency bands are combined as in a second embodiment described later.

The reliability calculation section 140 may obtain a reliability based on information indicating a difference or a ratio between the first local minimum value and the second local minimum value, respectively being the smallest one and the second smallest one of the local minimum values of the cost value. Alternatively, a reliability may be obtained based on information indicating a difference or a ratio between the first local maximum value and the second local maximum value, respectively being the largest one and the second largest one of the local maximum values of the cost value.

Alternatively, the reliability calculation section 140 may obtain a reliability based on the steepness of the change in the cost value relative to the change in the shift amount, within a given shift amount rage including the local maximum value or the local minimum value of the cost value.

Thus, the reliability can be obtained through various methods. The method of obtaining the reliability is not limited to these, and the reliability may be obtained through other methods or may be obtained through an appropriate combination between a plurality of methods.

When resonance frequencies f₁ to f_{N} of the first to the N-th bandpass filters BPF1 to BPFN satisfy fₖ<fₖ₊₁ (k being an integer satisfying 1≦k≦N−1), fHₖ≧fLₖ₊₁ may hold true where fHₖ and fLₖ₊₁ respectively represent an upper cutoff frequency of a k-th bandpass filter BFPk and a lower cutoff frequency of a k+1th bandpass filter BPFk+1, in the first to the N-th bandpass filters BPF1 to BPFN.

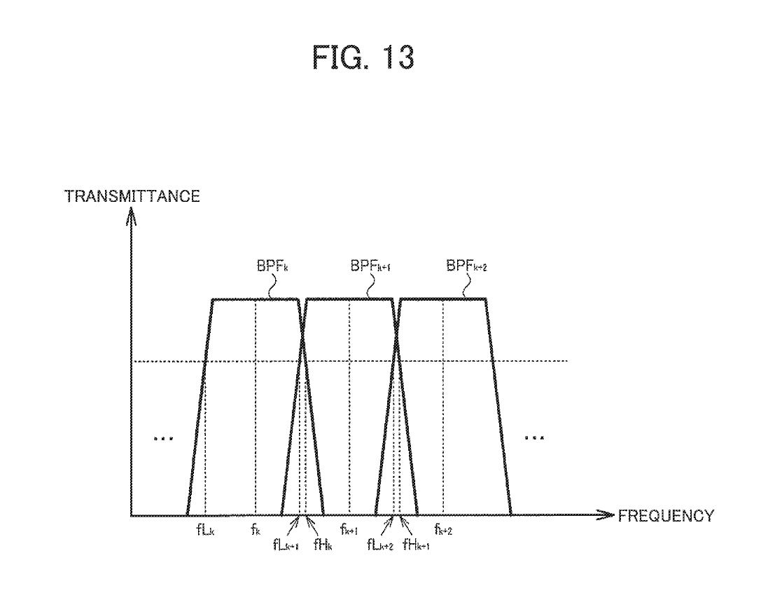

Thus, as illustrated in FIG. 13, the frequency bands may be set in such a manner that two adjacent bandpass filters have passbands overlapping with each other (at least have matching cutoff frequencies). Thus, one frequency (frequency band) is included in a passband of at least one of the bandpass filters, whereby the amount of disparity can be calculated with the frequency band well representing the feature of the subject, regardless of the value of such a frequency band. In other words, no lack of frequency band occurs, and thus stereo matching with higher versatility can be achieved.

When the reliabilities of all of the first to the N-th correlation calculation results are smaller than a given threshold value, the amount of disparity calculation section 160 may obtain an amount of disparity at the target pixel based on an amount of disparity obtained with a pixel other than the target pixel.

Thus, when an amount of disparity cannot be accurately obtained for a given target pixel within a flat area for example, information on the other pixel is used so that information with a low reliability needs not to be used. Thus, an amount of disparity at the target pixel can be obtained appropriately (with a certain level of accuracy). As used herein “the pixel other than the target pixel” is a pixel in the vicinity of the target pixel on the disparity map (on the first image) for example. Specifically, such a pixel may be a pixel with a distance to the target pixel not exceeding a given threshold value. Alternatively, subject recognition may be performed on the first image, and a pixel determined to correspond to the same subject and the same area as the target pixel may be used.

The weight setting section 150 may set the weight to 0 for ones of the first to the N-th correlation calculation results with reliabilities are smaller than a given threshold value.

Thus, the correlation calculation result (the cost functions in the present embodiment) with low reliabilities can be excluded in the later processes, whereby an amount of disparity can be accurately obtained and the amount of calculation can be reduced.

### 3. Second Embodiment

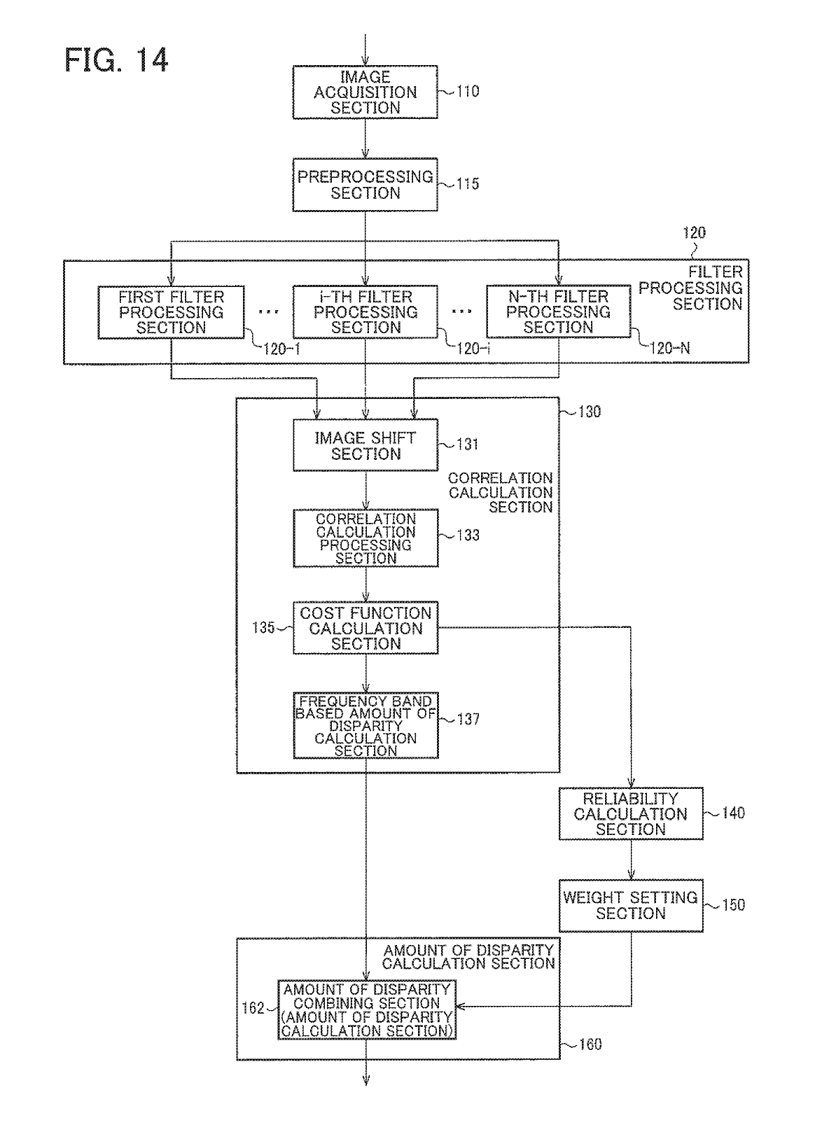

FIG. 14 illustrates an example of a configuration of an image processing device according to the second embodiment. The image processing device includes the image acquisition section 110, the preprocessing section 115, the filter processing section 120, the correlation calculation section 130, the reliability calculation section 140, the weight setting section 150, and the amount of disparity calculation section 160. The filter processing section 120, the reliability calculation section 140, and the weight setting section 150 are the same as those in the first embodiment, and thus a detailed description thereof is omitted.

The correlation calculation section 130 according to the second embodiment includes the image shift section 131, the correlation calculation processing section 133, the cost function calculation section 135, and a frequency band based amount of disparity calculation section 137. The amount of disparity calculation section 160 includes an amount of disparity combining section (amount of disparity calculation section) 162. The image processing device is not limited to the configuration illustrated in FIG. 14, and various modifications may be made with the components in the figure partially omitted or unillustrated components additionally provided.

The image shift section 131, the correlation calculation processing section 133, and the cost function calculation section 135 of the correlation calculation section 130 are the same as those in the first embodiment. In the present embodiment, the correlation calculation result is not the cost function of each frequency component obtained by the cost function calculation section 135, and is an amount of disparity obtained for each frequency component based on the cost function.

The detail of the method is the same as that in the first embodiment where the amount of disparity is obtained from the combined cost function, and a shift amount with the minimum value (maximum value) of the cost function may be obtained as the amount of disparity d. The frequency band based amount of disparity calculation section 137 obtains an amount of disparity from each of the first to the N-th cost functions, and thus obtains first to N-th amounts of disparity. The correlation calculation section 130 outputs the first to the N-th amounts of disparity thus obtained as the correlation calculation result.

The reliability calculation section 140 and the weight setting section 150 perform processes that are the same as those in the first embodiment. Thus, a weight is set for each frequency band. In the present embodiment, the weight is set for each of the first to the N-th amounts of disparity.

The amount of disparity calculation section 160 obtains an amount of disparity based on the first to the N-th amounts of disparity and the weights thus set. Specifically, the amount of disparity combining section 162 performs weighted averaging by using first to N-th amounts of disparity d1 to dN calculated by the frequency band based amount of disparity calculation section 137 and the weights w1 to wN set by the weight setting section 150. More specifically, this can be achieved by calculation in the following Formula (4).

$$\begin{matrix}[ Formula 4 ] & \\ {d}_{total} = \frac{1}{{\sum }_{i} {w}_{i}} {\sum }_{i} {w}_{i} {d}_{i} & ( 4 )\end{matrix}$$

As is apparent from the comparison between Formula (3) described above and Formula (4) described above, in the present embodiment, the calculation needs not to be performed for each shift amount k, and the final amount of disparity (combined amount of disparity dₜₒₜₐₗ) can be directly obtained by the calculation in Formula (4) described above.

Thus, in the present embodiment, the amount of disparity is obtained for each frequency band, and the weighted averaging is performed based on the reliability. Thus, the amount of disparity can be calculated without holding the cost functions of all the frequency bands. The calculation cost and the memory usage can be reduced because the cost functions of all the frequency bands need not to be held.

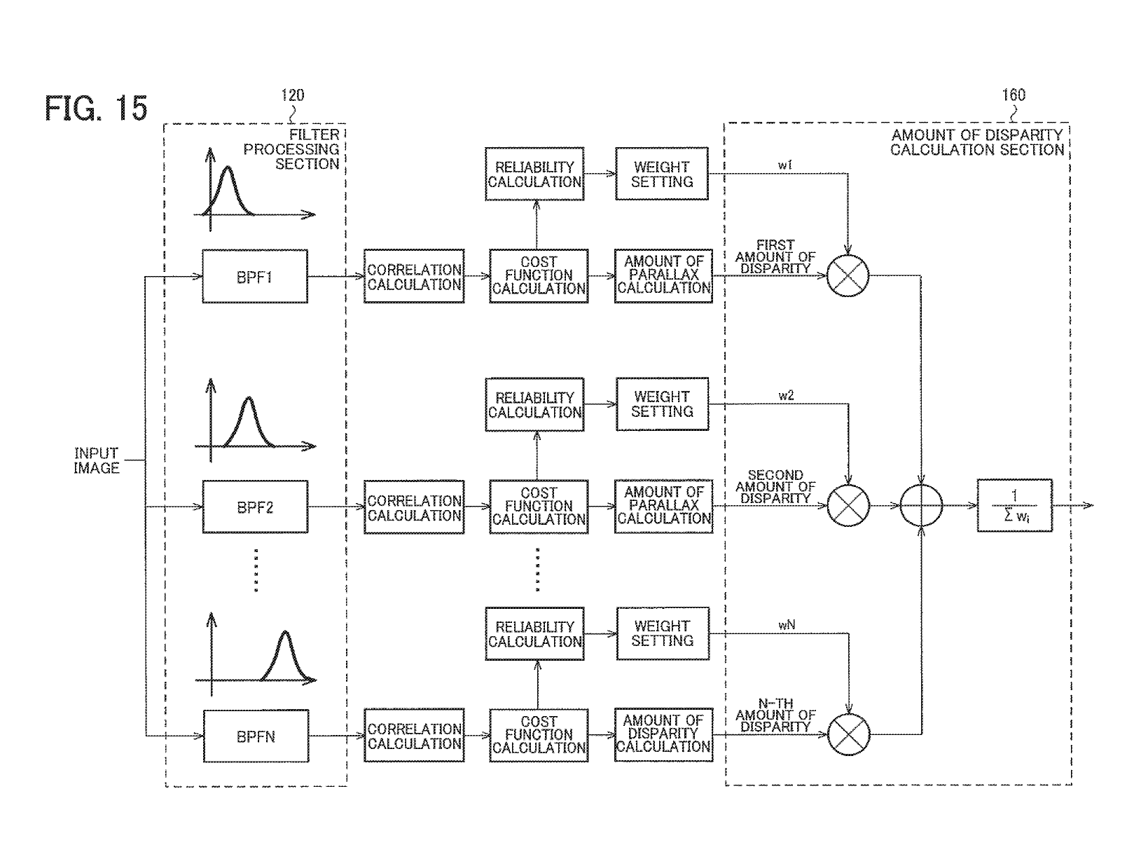

FIG. 15 is a schematic view illustrating the process according to the present embodiment described above. As is apparent from the comparison with the schematic views in FIG. 2 and FIG. 11, in the present embodiment, the amount of disparity is obtained for each frequency band as the correlation calculation result. Thus, the combining process performed by the amount of disparity calculation section 160 is a process for combining the amounts of disparity to obtain the final amount of disparity.

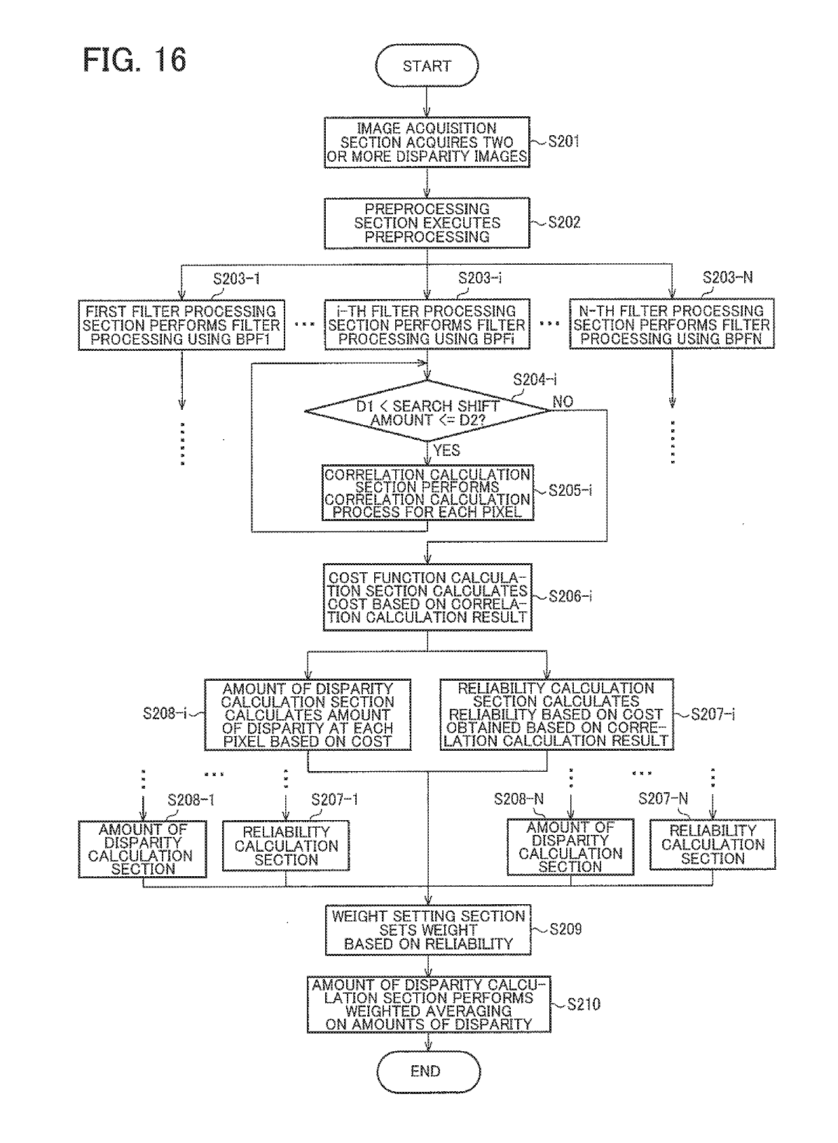

FIG. 16 is a flowchart illustrating the process according to the present embodiment. S201 to S207 are the same as S101 to S107 in FIG. 12, and thus detailed description thereof is omitted. In the present embodiment, a process for obtaining an amount of disparity S208 (S208-1 to S208-N) based on the cost function obtained in S206 (S206-1 to S206-N) is added as the process for each frequency band.

Weight setting (S209) is the same as S108 in FIG. 12. This embodiment may also be modified in such a manner that the weight setting process is performed for each frequency band.

The process for combining the amounts of disparity based on the first to the N-th amounts of disparity d1 to dN obtained in S208 and the weights w1 to wN obtained in S209 is performed (S210). Specifically, the process for obtaining the combined amount of disparity dₜₒₜₐₗ may be performed with Formula (4) described above. When there are a plurality of pixels for which the amount of disparity is to be obtained, the processes in FIG. 16 may be repeated for the number of times corresponding to the number of pixels, as in the first embodiment. For example, the processes in FIG. 16 may be repeated for the number of times corresponding to the number of all the pixels on the reference image.

In the present embodiment described above, the first to the N-th correlation calculation results are the first to the N-th amounts of disparity d1 to dN, which are amounts obtained for the frequency components based on the cost functions. The cost function is information in which the cost value, calculated by the correlation calculation, and the shift amount k are associated with each other. The amount of disparity calculation section 160 performs the weighted sum process using the weights w1 to wN set by the weight setting section, for the first to the N-th amounts of disparity d1 to dN, to obtain the amount of disparity (combined amount of disparity dₜₒₜₐₗ).

Thus, the final amount of disparity can be obtained by obtaining the amount of disparity for each frequency band as the correlation calculation result, and weighting and combining the amounts of disparity. The amount of disparity can be expressed with a small amount of data (for example, with a simple scalar), whereby the present embodiment requires an extremely small amount of memory for holding the correlation calculation result. Thus, the calculation cost and the memory usage can be reduced compared with the first embodiment.

The various modifications described above in the first embodiment may be also be applied to the present embodiment. For example, the weight setting section 150 may set the weight to be 0 for ones of the first to the N-th correlation calculation results determined to have reliabilities lower than a given threshold value.

Thus, the correlation calculation result (the amounts of disparity for each frequency band in the present embodiment) with a low reliability can be excluded from the calculation for obtaining the final amount of disparity (dₜₒₜₐₗ). In the method according to the present embodiment, an amount of disparity has a larger impact on the final amount, compared with the configuration where the cost functions are combined as in the first embodiment, no matter how small the weight of the amount of disparity may be. This is because the present embodiment featuring the direct calculation of the amount of disparity with Formula (4) described above includes no step of obtaining the minimum value (maximum value) of the combined cost function obtained by the combining as in the configuration of combining the cost functions. Thus, the amount of disparity can be more easily calculated with the weights set to be 0 for the amount of disparity for the frequency band with a reliability lower than the threshold value, and the frequency band with low reliability excluded from the process so that the process can be performed with amounts of disparity for the frequency bands with the highest reliabilities.

The image processing device and the like according to the present embodiment may include a processor and a memory. The functions of individual units in the processor may be implemented by respective pieces of hardware or may be implemented by an integrated piece of hardware, for example. The processor may include hardware, and the hardware may include at least one of a circuit for processing digital signals and a circuit for processing analog signals, for example. The processor may include one or a plurality of circuit devices (e.g., an IC) or one or a plurality of circuit elements (e.g., a resistor, a capacitor) on a circuit board, for example. The processor may be a CPU (Central Processing Unit), for example, but this should not be construed in a limiting sense, and various types of processors including a GPU (Graphics Processing Unit) and a DSP (Digital Signal Processor) may be used. The processor may be a hardware circuit with an ASIC. The processor may include an amplification circuit, a filter circuit, or the like for processing analog signals. The memory may be a semiconductor memory such as an SRAM and a DRAM; a register; a magnetic storage device such as a hard disk device; and an optical storage device such as an optical disk device. The memory stores computer-readable instructions, for example. When the instructions are executed by the processor, the functions of each unit of the image processing device and the like are implemented. The instructions may be a set of instructions constituting a program or an instruction for causing an operation on the hardware circuit of the processor.

The first and second embodiments, to which the present invention is applied, and modifications thereof have been described above. This should not be construed in a limiting sense however, and the present invention can be embodied with some components modified without departing from the scope of the present invention. Various inventions can be devised by combining a plurality of components disclosed in the first and second embodiments and the modifications thereof as appropriate. For example, some of the components described in the first and second embodiments and the modifications thereof may be omitted. In addition, components recited in different embodiments or modifications may be combined as appropriate. Terms that are accompanied by a broader term or a similar term at least once in the specification or the drawings can be replaced with such a term in any other part of the specification or the drawings. In this sense, various modifications and changes can be made without departing from the spirit of the present invention.

What is claimed is:

1. An image processing device comprising a processor comprising hardware,
the processor being configured to implement:
an image acquisition process for acquiring a plurality of images at least including a first image and a second image;
a filter process for extracting first to N-th (N being an integer equal to or larger than 2) frequency components from each of the first image and the second image, using first to N-th bandpass filters which pass first to N-th frequency bandwidths respectively;
correlation calculation for obtaining first to N-th correlation calculation results by performing correlation calculation with an i-th (i being an integer satisfying 1≦i≦N) frequency component in the first image and the i-th frequency component in the second image to obtain an i-th correlation calculation result at a target pixel;
a reliability calculation process for obtaining reliability of each of the first to the N-th correlation calculation results obtained;
a weight setting process for setting a weight of each of the first to the N-th correlation calculation results using the reliability; and
amount of disparity calculation for obtaining an amount of disparity between the first image and the second image at the target pixel based on the set weight and the first to the N-th correlation calculation results.

2. The image processing device as defined in claim 1,
the processor obtaining a corresponding pixel on the second image, the corresponding pixel being a pixel shifted from the target pixel on the first image by a set shift amount,
the processor obtaining the i-th correlation calculation result at the target pixel based on information corresponding to the target pixel in the i-th frequency component in the first image and based on information corresponding to the corresponding pixel in the i-th frequency component in the second image.

3. The image processing device as defined in claim 2,
the first to the N-th correlation calculation results being first to N-th cost functions,
each of the first to the N-th cost functions being information in which a cost value, calculated by the correlation calculation, and the shift amount are associated with each other.

4. The image processing device as defined in claim 3,
the processor obtaining a combined cost function by performing a weighted sum process using the weight, set by the weight setting process, on the first to the N-th cost functions, and obtaining the among of disparity based on the combined cost function.

5. The image processing device as defined in claim 2,
the first to the N-th correlation calculation results being first to N-th amounts of disparity, each of the first to the N-th amounts of disparity being an amount of disparity obtained for a corresponding one of the frequency components based on a cost function obtained by associating a cost value, calculated by the correlation calculation, with the shift amount.

6. The image processing device as defined in claim 5,
the processor obtaining the amount of disparity by performing a weighted sum process, using the weight set by the weight setting process, on the first to the N-th amounts of disparity.

7. The image processing device as defined in claim 3,
the processor obtaining the reliability based on information on a difference or a ratio between a first local minimum value and a second local minimum value, the first local minimum value and the second local minimum value respectively being a smallest one and a second smallest one of local minimum values of the cost value, or based on information on a difference or a ratio between a first local maximum value and a second local maximum value, the first local maximum value and the second local maximum value respectively being a largest one and a second largest one of local maximum values of the cost value.

8. The image processing device as defined in claim 3,
the processor obtaining the reliability based on a steepness of a change in the cost value relative to a change in the shift amount, within a given shift amount range including a local maximum value or a local minimum value of the cost value.

9. The image processing device as defined in claim 1,
the first to the N-th bandpass filters having resonance frequencies f₁ to f_{N} satisfying fₖ<fₖ₊₁ (k being an integer satisfying 1≦k≦N−1),
fHₖ≧fLₖ₊₁ being satisfied where fHₖ represents an upper cutoff frequency of a k-th bandpass filter in the first to the N-th bandpass filters and fLₖ₊₁ represents a lower cutoff frequency of a k+1th band pass filter.

10. The image processing device as defined in claim 1, the processor obtaining, when the reliability of each of the first to the N-th correlation calculation results is smaller than a given threshold value, the amount of disparity at the target pixel is obtained based on the amount of disparity of a pixel other than the target pixel.

11. The image processing device as defined in claim 1, the processor setting the weight to be 0 for a correlation calculation result, the reliability of which is smaller than a given threshold value, among the first to the N-th correlation calculation results.

12. An endoscope device comprising the image processing device as defined in claim 1.

13. The endoscope device as defined in claim 12, the first image and the second image each being an in vivo image.

14. An image processing method comprising:
performing a process for acquiring a plurality of images at least including a first image and a second image;
extracting first to N-th (N being an integer equal to or larger than 2) frequency components from each of the first image and the second image, using first to N-th bandpass filters which pass first to N-th frequency bandwidths respectively;
obtaining first to N-th correlation calculation results by performing correlation calculation with an i-th (i being an integer satisfying 1≦i≦N) frequency component in the first image and the i-th frequency component in the second image to obtain an i-th correlation calculation result at a target pixel;
obtaining reliability of each of the first to the N-th correlation calculation results obtained;
setting a weight of each of the first to the N-th correlation calculation results using the reliability; and
obtaining an amount of disparity between the first image and the second image at the target pixel using the set weight and the first to the N-th correlation calculation results.
